(19) United States
(12) Patent Application Publication
(10) Pub. No.: US 2011/0250272 A1
(43) Pub. Date: Oct. 13, 2011

(54) SOLID, ORODISPERSIBLE AND/OR DISPERSIBLE COMPOSITION, WITHOUT AN EXCIPIENT OF KNOWN EFFECT AND ITS PROCESS OF PREPARATION

(75) Inventors: Jérôme BESSE, Saint Jean D'Illac (FR); Laurence Besse, Saint Jean D'Illac (FR); Julien Pournin, Talence (FR)

(73) Assignee: Galenix Innovations, Saint Jean D'Illac (FR)

(21) Appl. No.: 13/169,836

(22) Filed: Jun. 27, 2011

Related U.S. Application Data
(63) Continuation of application No. 12/201,078, filed on Aug. 29, 2008, now abandoned.
(60) Provisional application No. 60/973,216, filed on Sep. 18, 2007.

(30) Foreign Application Priority Data
Aug. 31, 2007 (FR) 07 57295

(51) Int. Cl. A61K 9/20; A61K 31/353; A61K 31/445; A61P 25/28; A61P 9/00; A61P 9/12; A61P 25/18; A61K 9/14; A61K 31/519

(52) U.S. Cl. 424/465; 424/400; 514/456; 514/319; 514/259.41

(57) ABSTRACT

The present invention relates to a solid, orodispersible and/or dispersible composition comprising (a) from 0.1 to 59% by weight of at least one active substance with particle size no exceeding 50 μm; (b) from 40 to 99% by weight of at least one diluent without known effect, non water-soluble; (c) from 0.1 to 15% by weight of at least one disintegrating agent; and (d) from 0.05 to 10% by weight at least of one sweetening agent with particle size not exceeding 50 μm, percentages by weight being expressed compared to the total weight of the aforementioned composition. The present invention also relates to the use of said composition as a drug, a food supplement or in cosmetics and a method of preparation of an orodispersible and/or dispersible compound implementing said composition.

### CROSS-REFERENCE TO RELATED APPLICATIONS

This application is a continuation application of U.S. application Ser. No. 12/201,078, filed on Aug. 29, 2008, and claims priority to U.S. Provisional Application No. 60/973,216 filed Sep. 18, 2007, and to French Application No. 07 57295, filed Aug. 31, 2007.

### TECHNICAL FIELD

The present invention refers to the field of pharmaceutical, cosmetic, parapharmaceutical, neutraceutical, food and agroalimentary compositions, as well as their methods of preparation.

More specifically, the present invention relates to a solid composition, without an excipient of known effect, disintegrating in the mouth or under the tongue in less than 20 sec, or dispersing in water without prior agitation in less than 3 min, obtained according to a simple mode of preparation not altering the physicochemical and pharmacokinetic properties of the active substance(s) present in the composition. The active substance(s) present in the composition may be of hydrophilic, hydrophobic or amphiphilic nature, their average particle size distribution does not advantageously exceed 50 μm. The composition free from excipient of known effect, the object of this invention does not require any coating or lamination to mask the had taste or the bitterness of certain, active substances.

The simile mode of preparation guarantees the industrial feasibility of a homogeneous, stable and non-friable composition.

### BACKGROUND ART

For better comfort of the patient and better observance, various compositions with immediate release disintegrating in the mouth were placed on the market.

Therefore, there are preparations in the form of oral lyophilizate. These forms have the advantage of disintegrating quickly in spite of the poor solubility of the active substance but the manufacturing process of such a form requiring a freeze-drying step is complex and expensive. The compositions obtained are, in general, friable and require a protective conditioning of moisture to guarantee their integrity. In addition, once disintegrated, the grains dispersed in the mouth confer a granular and farinaceous sensation which is particularly uncomfortable.

To avoid the freeze-drying step, it was proposed to make orodispersible tablets. Many patented technologies (OraSolv, DuraSolv, Flash Dose, WowTab, FlashTab, OraQuick . . . ) and manufacturing processes around this galenic form are numerous, they try to solve 3 main problems
obtaining a time of disintegration of the tablet in the mouth, without contribution of water, less than 3 min as recommended in the various pharmacopoeias in force. To this problem, is added the fact that, from one individual to another, the quantity of saliva present in the mouth, is variable;
obtaining tablets with specifications of acceptable hardness and friability allowing to preserve good properties of disintegration;
masking of bitterness or bad taste of many active ingredients.

To solve the problem of disintegration, several techniques are described in the previous art, such as the use of soluble excipients, of super-disintegrating agents or of effervescent pairs.

The international application WO 00/27357 described a multiparticle orodispersible tablet able to disintegrate in less than 40 seconds, containing polyol.

The US patent application n^{o}2003147947 describes a composition containing granule-based lactose and co-dried starch, (StarLac™).

OraSolv® Technology from Cima Labs produces a tablet that disintegrates quickly in the mouth by using low effervescence.

To solve the problems of friability, the international application WO 2004/043440 uses a polysaccharide produced from a yeast which is the pullulan and the tablets, of which the friability is less than 1%, are obtained by a process either of freeze-drying or of fusion-moulding.

To increase the hardness of the orodispersible tablets, U.S. Pat. No. 6,024,981 uses a diluent not adapted to direct compression, the orodispersible tablets therefore obtained having a hardness ranging between 15 and 50 N.

The patent application EP 1156786 teaches tablets that are slightly friable thanks to the use of a lubricant on the surface of the tablets.

For the masking of taste, FlashTab® technology described by Ethypharm in the patent application EP 1441704 relates to orodispersible tablets containing coated active ingredients that are microcrystals or microgranules.

The major disadvantage of these compositions is the presence of excipients with known effect which, consequently, involve risks of intolerance among certain categories of patients. In the same way, these various patented technologies require conditions of manufacture and storage of the products in a controlled atmosphere taking into account the strongly hygroscopic character of the powders generated by these processes and compositions.

To resolve this disadvantage, the international application WO 2005/034921 teaches an orodispersible composition based on a neurological agent not very soluble or insoluble using, preferably, as diluent sugars but possibly being able to include as diluent microcrystalline cellulose or dextrate, excipients of known effect. However, to guarantee the stability of the composition a stabilizing agent is necessary. In addition, the preferred embodiment integrates a step of wet granulation.

International application WO 2004/091585 describes a composition of oral disintegration based on silicified microcrystalline cellulose such as Prosolv® This type of microcrystalline cellulose guarantees the stability of the composition but is particularly expensive. Moreover, the silicified microcrystalline cellulose cannot be used with all the active substances due to incompatibility.

There is a need, in the state of the art, to develop an orodispersible composition and a simple method of preparation of the latter not presenting the known technical disadvantages for the orodispersible compositions of the previous art.

### PRESENTATION OF THE INVENTION

The work of the Applicant made it possible to develop an orodispersible solid composition without an excipient of known effect and non-hygroscopic and its method of preparation, both making it possible to deal with the technical disadvantages cited above.

Moreover, the solid composition, orodispersible object of this invention is remarkable for the fact that it applies to various technical fields. Indeed, this composition can be a pharmaceutical, cosmetic, parapharmaceutical, neutraceutical, food or agroalimentary composition.

The solid composition object of this invention is also remarkable through the fact that it shows the appropriate specifications not only for an orodispersible composition but also for a dispersible composition. The composition object of this invention therefore constitutes an orodispersible and/or dispersible composition.

The solid, orodispersible and/or dispersible composition object of this invention appears as an uncoated tablet, disintegrating quickly in the mouth or under the tongue, or disintegrating quickly in a glass of water without alteration of the pharmacokinetic properties of the active substance present in the composition, the aforementioned tablet being obtained according to a simple mode of preparation. The active substance present in the composition can be of an hydrophilic, hydrophobic or amphiphilic nature, their average particle size not exceeding, advantageously 50 μm. Advantageously, the active substance (s) is/are found in a micronized form. Thus, any active substance present in the composition according to the invention can be subjected to a process of prior micronizing, no other step of preliminary treatment such as a deposit on an inert support or a granulation having been used. Thus, the active substance(s) present in the composition is/are not present in a multiparticle form.

The characteristics of the solid, orodispersible and/or dispersible composition according to the invention were attained thanks to the development of a particular combination of an active substance with excipients presenting particular characteristics and the use of a manufacturing process including steps of successive dilutions then a step of direct compression.

The composition object of this invention is free from excipient of known effect and is inexpensive. The particles of active substance require neither coating, nor laminating to mask their unpleasant flavours. The composition object of this invention advantageously does not include a stabilizing agent.

The simple method of preparation, object of this invention, guarantees the industrial feasibility of a homogeneous, stable and non-friable composition thanks to a process including several steps of dilution, then a step of direct compression.

Within the scope of this invention, one understands by “homogeneous composition”, any composition which satisfies the test of uniformity of content of single-dose preparations as described in the European Pharmacopoeia in force, namely an individual content of each unit ranging between 85 and 115% of the average content.

Within the scope of this invention, one understands by a “stable compositions” any composition preserving these initial characteristics after being subjected to a stability study to ICH standards.

Within the scope of this invention, one understands by “non-friable composition” any composition which satisfies the test of friability described in the European Pharmacopoeia, namely a loss of maximum mass of 1%.

The present invention concerns, more particularly, a solid, orodispersible and/or dispersible composition, without an excipient of known effect, comprising:

a) from 0.1 to 60% by weight of at least one active substance with particle size not exceeding 50 μm;

b) from 40 to 99% by weight of at least one thinner, without known effect, non water-soluble;

c) from 0.1 to 15% by weight of at least one disintegrating agent; and

d) from 0.05 to 10% by weight of at least one sweetening agent of particle size not exceeding 50 μm, the percentages by weight being expressed compared to the total weight of the aforementioned composition.

Within the scope of this invention, one understands by “orodispersible composition”, an uncoated tablet, intended to be placed in the mouth or under the tongue where it disperses quickly before being swallowed (European Pharmacopoeia 5.8). An orodispersible tablet disintegrates or dissolves, in water, at 37° C., in less than 3 minutes. Better still, thanks to the invention, it dissolves in less than 2 minutes, notably in less than 1 minute, in particular, in less than 30 seconds and, more particularly, in less than 20 seconds.

Within the scope of this invention, one understands “dispersible composition”, an uncoated tablet intended to be dissolved in water before administration to form a homogeneous dispersion (European Pharmacopoeia 5.8). A dispersible tablet breaks up in water at 15-25° C. in less than 3 min, without prior agitation. Better still, thanks to the invention, it breaks up in less than 2 minutes, notably in less than 1 minute, in particular, in less than 30 seconds and, more particularly, in less than 20 seconds. Homogeneous dispersion obtained must be able to go through a sieve with a nominal mesh opening of 710 μm.

Within the scope of this invention, one understands by “without an excipient of known effect” any composition not requiring any precaution for use for certain particular categories of patients. Will be in particular excluded from the composition according to the invention, the polyols such as mannitol, xylitol, sorbitol, maltitol, maltitol syrup, isomalt, aspartam etc. . . . and any other excipient cited in the list of excipients of known effect and likely to generate intolerances. Therefore, also excluded from the composition according to the invention are boric acid and its salts with an amount higher than 3 mg/kg/day; wheat starch; bronopol with an amount higher than 0.05 (percentage by weight expressed as a ratio of the total weight of the composition) benzalkonium chloride; organomercury compounds; ethanol with an amount higher than 0.05 g/day; formaldehyde with an amount higher than 0.05% (percentage by weight expressed as a ratio of the total weight of the composition); fructose; galactose; glucose; glycerol with an amount higher than 1 g/take or higher than 3 a/24 hours; peanut oil; castor oil and its derivatives; soya bean oil and its derivatives; sesame oil; lactose; paraformaldehyde with an amount higher than 0.5% (percentage by weight expressed as a ratio of the total weight of the composition); polyethyleneglycol with an amount higher than 2 q/take or higher than 6 g/day; phenylalanine; potassium; saccharose; sodium with an amount higher than 200 mg/day for compositions intended for adults; invert sugar; sulphites and metabisulphites; tartrazine and azo dyes.

Any value quantified as “X” provided within the scope of this invention is understood, taking into account experimental errors, as X±Y with Y being between X/100 and X/10.

The orodispersible and/or dispersible solid composition, object of this invention comprises at least one active substance. In a variation of the invention, the aforementioned composition comprises a mixture of two, three, our or more different active substances.

The active substance (s) comprised in the composition according to the invention may be any active ingredient having an activity in the pharmaceutical, parapharmaceutical, cosmetic, neutraceutical fields, for food or agroalimentary additives. These active substances can be hydrophilic, lipophilic or amphiphilic. Advantageously, their average particle size distribution does not exceed 50 μm, in particular 30 μm, and, most particularly, 20 μm.

Within the scope of this invention, one understands by “particle size” of a powder according to the invention, the average size of the particles which constitute it. The average size of the particles can be measured by any known conventional technique known in itself. In particular, one skilled in the art may have recourse to a measurement of particle size by way of a laser of type Beckman Coulter® or Malvern®.

The active substance(s) implementable in the composition according to the invention may be selected from among the active ingredients typically used in pharmacotherapy and in particular in the following pharmacotherapeutic families: allergology, anaesthesia/resuscitation cancerology and haematology, cardiology and angiology, contraception and termination of pregnancy, dermatology, endocrinology, gastro-entero-hepathology, gynaecology and obstetrics, immunology and transplantation drugs, the study of infections and parasitology, metabolism, diabetes and nutrition, neurology/psychiatry, ophthalmology, otorhinolaryngology, pneumology, rheumatology, stomatology, toxicology, urology/nephrology, as well as among analgesic, anti-pyretic and antispasmodic drugs, anti-inflammatory drugs, the products of diagnosis, haemostatics and the blood treatment products and derivatives.

In particular, the active substance(s) may be selected from the group consisting of active ingredients useful or used in the treatment/prevention and/or prophylaxis of a pathology or a disorder affecting the central nervous system, of a gastric pathology or disorder or of a inflammatory pathology or disorder and any other group of active ingredients requiring ambulatory treatment.

Advantageously, the active substance(s) may be selected from the group consisting of anti-inflammatory drugs and in particular non-steroid anti-inflammatory drugs, anti-epilepsy, antiparkinson, antimyasthenic, antispastic, antimigraine, antiepilepsy, ataractics, antidepressants, psychotropic drugs, anticholinesterasics, antagonists of NMDA receptors, antiacids, gastric antisecretory, antagonists to dopamine, antispasmodics, antimigraine, choleretics, hepatotropic, laxatives, derivatives of vitamin A, antivirals, anti-cancer, analgesics, antiulceric, antipsoriasis, antibiotics, inhibitors of cyclo-oxygenase (COX inhibitors), sexual hormones (for example, antioestrogens, oestrogens, pro-gestatives, androgenes and antiandrogenes).

By way of examples and in a non-exhaustive way, the active substance(s) may be selected from the group consisting of acetazolamide, dipropionate of alclometasone, acyclovir, adapalene, the adrenergic agonist. β-3, alclomethasone dipropionate, alosetron, alprostadil, alprozolam, benzodiazepine alprozolam, amcinonide, ameleine, 5-aminolevulinic acid, amoxicilline, androstanolone, Δ-4-androstenedione, aripipazole, bamethan sulphate+escine, betamethasone valerate, betamethasone dipropionate, bufexamac, buprenorphine, buspirone, caffeine, calcipotriol monohydrate, capravirine, caspofongine acetate, cefaclor, cetrimonium bromide, cilomilast, clobetasol propionate, clonazepam, clonidine, cloxacilline, coenzyme Q10, crilanomere, cyclosporine, cyproterone acetate, darifenacine, desogestrel, 3-keto-desogestrel, desonide, dexmedetomidine, dexpanthenol, diazepan, diclofenac, diflucortonole, difluprednate, diphenydramine chloride, donepezil, eletriptan, econazole nitrate, eptazocine, estradiol and its derivatives estradiol hexahydrobenzoate, estradiol undecylate, valerate estradiol, ethinyl estradiol), erythromicine, esomeprazole, etoricoxib, ezetimide, fenoprofen, fentanyl, flubiprofen, flumetasone, fluocinolone pivalate, fluocinodine, fluocortolone, fluocortolone hexanoate, fluocortolone pivalate, growth hormone, hydrocortisone, hydrocortisone acetate, ibacitabine, ibuprofene, imiquimod, indometacine, insulin, interferon α, isosorbide dinitrate, isotretinoine, ketoconazole, ketoprofen, ketorolac, ketotifen, lansoprazole, levonorgestrel in association with ethinylestradiol or estradiol, lidocaine, lorazepan, mecamylamine, medindolol, melagatran, mementine, methylphenidate, metronidazole, miconazole, miconazole nitrate, minoxidil, mitiglinide, molsidomine, naloxone, naproxene, nebivolol, nestorone, nicotine, nicotine S(−), niflumide acid, nitro-glycerine, norethisterone acetate, norethisterone enanthate, 7α-methyl-19-nortesterone, omapatrilat, omeprazole, orzel, oxybutinine, parecoxib, penciclovir, pergolide, benzoyl peroxide, phlorgiucinol, physostigmine, piroxam, piroxicam, pitavastatine, povidone-iodine, prazosin, prednisolone, progesterone, promestriene, prostaglandins, pyrazonibutasone, raloxifene, risperidone, rosuvastatine, rotigotine, roxithromycine, salbutamol, scopolamine, selegiline, substance P antagonist, sulfacetalmide, tazarotene, tegaserod, testosterone, dihydrotestosterone testosterone, cyclohexylmethylcarbonate, testosterone propionate, thiatolserine, thinylestradiol+norelgestromin, triamsinolone, trinitrine, triptans such as frovatriptan, naratriptan, zolmitriptan, rizatriptan, sumatriptan, eletriptan and almotriptan, tretinoine and isotretinoine, triclocarban, trimethadione, tulobuterol, valacyclovir, valdecoxib, valproic acid, vidarabine monophosphate, viozan, voriconazole, salts and mixtures thereof.

The active substances implemented within the scope of this invention may be also chosen among the actives typically used in cosmetics, parapharmacy and for food additives. The contents of these actives are those classically used in the fields considered.

Among the cosmetic and parapharmaceutical actives, one can quote emollients, wetting agents, pigments and dyes, anti-wrinkle agents (like retinol), anti-fungal agents, anti-acne agents, softening agents, perfumes, vitamins and mixtures thereof.

Among the actives for food additives, one can quote vitamins such as vitamins A, B, E, C, B1, B2, B3, BE, B9, B12, B8H, B5 . . . ; minerals such as calcium, phosphorus, iron, magnesium, zinc, iodine . . . ; carotenoids such as alpha-carotene, beta-carotene, gamma cartene, lutein, zeaxanthine, cryptoxanthine, lycopene . . . ; phyto-oestrogens such as isoflavones (for example, genisteine, diadzeine, biochanine A, formononetine); lignanes for example, enterolactone, enterodiol); coumestanes (for example, coumestrol); vegetable extracts and in particular extracts of fennel, heather, blackcurrant, grape pips, rockweed, ginseng, green coffee, ginger, pectin of apple, . . . ; oils such as oil of evening primrose, wheat germ, marrow pips . . . ; clays such as diosmestite, montmorillonites . . . ; ferments, yeasts and mixtures thereof.

Within the scope of this invention, it is clear that a mixture of active substances covers not only a mixture of active substances of the same group (i.e. pharmaceutical actives, cosmetics actives, food additive actives) but also a mixture of active substances taken from at least two of the groups cited above.

The active substance (s) implemented in the composition according to the invention may be natural, chemical, synthetic or biological origin. Indeed, the active substance(s) implemented in the composition according to the invention may be obtained by chemical synthesis or genetic engineering processes well-known to one skilled in the art. The active substance(s) implemented in the composition according to the invention may be used as such after recovery of the medium in which they are in a natural state or not, particularly via an extraction. The active substance(s) implemented in the composition according to the invention may also be used after a preliminary transformation such as purification, dilution, filtration, concentration, drying, freeze-drying or a process as described in the patent application FR 03 02802.

One skilled in the art will know according to the active substance (or of the mixture of active substances) implemented what quantity of active substance has to be used and this according to the final use of the composition according to the invention. Therefore, the quantity of active substance(s) is included in the composition according to the invention between 0.1 and 60% by weight, between 0.1 and 59 a by weight, notably between 0.5 and 30% by weight, in particular between 0.75 and 10% by weight and, more particularly, between 1 and 5% by weight compared to the total weight of the composition.

A diluent without known effect is used to supplement the solid pharmaceutical composition, orodispersible and/or dispersible, object of this invention until obtaining a predetermined total volume containing the selected quantity of the active substance.

The diluent without known effect in the composition according to the invention has sufficient binding properties exempting the use of a binder such as povidone, starch derivatives, polyvinyl alcohol or any other binder known to one skilled in the art. Therefore, in an advantageous way, the solid composition, orodispersible and/or dispersible according to the invention does not include a binder.

The diluent without known effect implemented within the scope of this invention has good flow and compression properties. As an example, this diluent has a flow less than 10 sec and a bulk volume below 20 ml.

The diluent without known effect implemented within the scope of this invention represents from 40 to 99% by weight, notably from 50 to 95% by weight and, in particular, from GO to 90% by weight compared to the total weight of the composition according to the invention.

The diluent without known effect implemented within the scope of this invention is a diluent, without known effect, non water-soluble, selected from the cellulose powder, non-silicified microcrystalline cellulose, the micro-crystalline cellulose with low water content (i.e. microcrystalline cellulose having a quantity of water lower than 1.5% by weight compared to the total weight of microcrystalline cellulose), cellulose acetate di- or tri-basic calcium phosphate, calcium sulphate, dextrates, dextrins, hydrogenated vegetable oils, glyceryl palmitostearate, polymethacrylate. These agents can only be used on their own or mixed and in particular in a mixture of two, three, four or more. The microcrystalline cellulose is selected for preference.

By “disintegrating agent”, one understands any substance or any mixture of these contributing essentially to the mechanical characteristics for the disintegration in a aquaeous medium of the tablets manufactured starting from the solid composition object of this invention.

The disintegrating agent represents from 0.1 to 15% by weight, notably from 1 to 10% by weight and, in particular, from 2 to by weight compared to the total weight of the composition.

The disintegrating agent implemented within the scope of the composition according to the invention is selected from among crospovidone (or cross-linked polyvinylpyrrolidone), croscarmellose (or cross-linked sodium carboxymethyl cellulose), sodium starch glycolate, sodium alginate, hydroxypropyl cellulose, starch, pre-gelatinized starch, derivatives and mixtures thereof. These agents can be used on their own or mixed and in particular in mixtures of two, three, four or more. The crospovidone and the croscarmellose are preferred disintegrating agents within the scope of this invention.

By “sweetening agent”, one understands any substance or any mixture of substances likely to considerably improve compliance of this composition, and more particularly any substance or any mixture of substances without known effect able to mask the unpleasant taste and the bitterness of certain active substances.

To be more effective and to therefore avoid requiring the coating or the film-coating of the active substance, the sweetening agents present a particle size lower than 50 μm, better and lower than 30 μm, still better and lower than 10 μm. Thanks to the use of an sweetening agent of particle size lower than 50 μm distributed in a homogeneous way, the use of a coating or film-coating agent is not necessary to mask unpleasant flavours of certain active substances.

The composition according to the invention can comprise a sweetening agent or a mixture of sweetening agents and in particular a mixture of two, three, four or more sweetening agents, since the percentage by weight of the sweetening agent or the mixture of sweetening agents goes from 0.05 to 10% by weight, notably from 0.1 to 5% by weight and, in particular, from 0.5 to 2.5% by weight compared to the total weight of the composition.

The sweetening agent(s) is (are) advantageously chosen among the sugar substitutes and, more particularly among gluconate, potassium acesulfame, sodium saccharinate, lithium saccharinate, cyclamate, or mixtures thereof. Indeed, within the scope of this invention, one prefers the sugar substitutes not metabolized in the organism with the natural sweetening substitutes and as such, to avoid adding one excipient with known effect in the composition according to the invention.

In order to supplement the organoleptic effect of the sweetening agent(s), the composition according to the invention can also comprise an flavouring agent or a mixture of flavouring agents. Therefore, according to a preferential embodiment of the pharmaceutical composition according to the invention, the organoleptic characteristics are still improved by the addition of this (or these) flavouring agent (s). Therefore, the composition according to the invention can also comprise from 0.005 to 10% by weight, notably from 0.05 to 8% by weight, in particular from 0.5 to 6% by weight and, more particularly from 0.75 to 4% by weight of a flavouring agent or a mixture of flavouring agents, compared to the total weight of the composition.

Preferably, the flavouring agent(s) is (are) chosen among the natural flavours, the natural identical flavours or the artificial flavours. In particular, the flavouring agent(s) is (are) selected from among a fruit flavour, a mint flavour, an anis flavour, a honey flavour, a vanilla flavour, a tea flavour and a verbena flavour. More particularly, is(are) used within the scone of this invention the following flavouring agent (s): apricot, apricot-orange, citrus fruits, pineapple-coconut, anis, banana, cocoa, caramel, caramel-fruit, blackcurrant, cherry, Morello cherry, cherry-raspberry, lemon, lime, orange oil, flower of orange tree, mill, raspberry, passion fruit, fruits of the forest, fruits of the orchard, red fruits, red/caramel fruits, grenadine, gooseberry, orange juice, tangerine, mango, mint, peppermint, mint-eucalyptus, honey, mirabelle plum, blackberry, bilberry, grapefruit, peach, pear, apple, plum, orange pulp, grape, liquorice, rosemary-orange tree, tea, vanilla, verbena or violet.

Advantageously, the flavouring agent (or the mixture of flavouring agents) is absorbed on a suitable support before being incorporated in the pharmaceutical composition according to the invention, in the shape of a powder of the pre-impregnated support. Any type of conventional support in pharmaceutical field can be used for the flavouring agent, such as for example silica, starch or cellulose powder.

The composition according to the invention can comprise one or more flowing agents guaranteeing a homogeneous distribution of the active substance or mixture of active substances within the composition and a good flow of the final mixture.

The flowing agent or the mixture of flowing agents re resents from 0.001 to 1% by weight, notably from 0.005% to 0.8% by weight and, in particular, from 0.01% to 0.5% by weight compared to the total weight of the composition.

Among the flowing agents usable within the scope of this invention, one can quote colloidal silica, silica dioxide, magnesium or calcium silica and talc. Within the scope of this invention, colloidal silica is preferentially selected.

Lastly, the composition according to the invention can comprise moreover a lubricant agent or a mixture thereof. At the time of manufacture of the tablet, one adds a quantity appropriate of lubricant agent or a mixture of lubricant agents at the external layer of the latter and in particular to the final mixture of the composition according to the invention right before subjecting it to a direct compression.

Preferably, the suitable quantity of the lubricant agent goes from 0.01 to 2% by weight, notably from 0.05 to 1.5% by weight and, in particular, from 0.1 to 1% by weight compared to the total of the composition.

The lubricant agent(s) is (are), advantageously, chosen among magnesium stearate, stearic acid and its derivatives, calcium stearate, sodium stearate, sodium stearyl fumarate, sodium acetate, sodium oleate, the sodium chlorate sodium benzoate, glyceril behenate, polyethylene glycol, talc and hydrogenated vegetable oils. The sodium benzoate can be also used as preservative and anti-bacterial. The magnesium stearate is one of the lubricant agents more preferentially used in the composition according to the invention.

Moreover, the composition can comprise a humectant or a mixture of humectants. One understands, within the scope of this invention, by “humectant”, any substance, in solid form, modifying the surface tension of water on the active substance in order to increase the wettability of the aforementioned active substance in aqueous medium.

The humectant or mixture of humectants represents from 0.01 to 5% by weight, notably from 0.05 to 2% by weight and, in particular, from 0.1 to 1% by weight compared to the total weight of the composition.

The humectant is from among the dodecylsulphate, sodium (SLS), a ester of polysorbitan, such as monooleate, monolaurate, monopalmitate, monostearate esters, esters of sorbitan, the polyoxyethylenes ethers, the sodium dioctylsulphosuccinate (DOSS), lecithin, sodium docusate, and mixtures thereof. Indeed, these agents can only be used alone or in association of two, three or four or more agents. Sodium lauryl sulphate is preferentially selected.

The composition according to the invention can also include other excipients of additive type such as colouring agents, salivation agents, preservatives, pH regulators, antioxidants etc. . . . One skilled in the art knows which type of compounds to use for each class of excipients above and in what proportion, the only constraint being to choose excipients without known effect. Moreover, among the components of the composition according to the present invention except for the active substance(s), at most 10%, in particular at most 6% and, in particular, at most 4% of these components are water-soluble. Lastly, among the insoluble components of the composition according to the present invention except for the active substance(s), at most 30%, at most 20%, notably at most 10%, in particular at most 5% and, more particularly, at most 2% of these components are inorganic.

Preferably, the pharmaceutical composition according to the invention is particularly adapted to an administration orally and, particularly, adapted to an oral and sublingual ambulatory use and/or to a use after dispersion. Therefore, the orodispersible and/or dispersible, solid composition according to the invention arises, advantageously, in forms of posological unit-dose packages containing acceptable quantities of active substances in particular acceptable pharmaceutical substances. Such quantities extend from 0.05 to 2000 mg, notably from 0.1 to 1000 mg, in particular from 1 to 500 mg and, more particularly, from 1 to 200 mg of active substances per dosage unit.

The preferred unit dosage form within the scope of this invention is a preparation conditioned in the form of tablets and, more particularly, of orodispersible and/or dispersible tablets. This tablet can be mono-layered resulting from only a single homogeneous mixture or multi layered resulting from several mixtures. Provided that, within the scope of multi-layered or with dual nucleus tablets, each mixture meeting the characteristics of the invention will constitute one of the layers of the tablet. In addition to the conventional tablet, the present invention can in particular relate to double-layered or tri-layered tablets. Moreover, the unit dosage form can be either such a tablet in itself, or a suitable number of such tablets. Such unit dosage forms are particularly adapted for one (or several) daily take(s) according to the application and in particular according to the therapy, of the phase of the therapy or other.

The present invention also relates to the use of the solid, orodispersible and/or dispersible composition according to the present invention such as previously defined as a drug, a food supplement or in cosmetics and thus a solid, orodispersible and/or dispersible composition according to the present invention such as previously defined, for use as a drug, a food supplement or in cosmetics.

From the qualitative and quantitative composition in active substance and in excipients described in the invention, a final formulation in the form of tablets can be easily prepared by a simple process of successive dilutions then direct compression of the mixture of the active substance and excipients, whose pharmacotechnical characteristics optimal. Preferably, a tablet in conformity with the invention has a breaking strength ranging between 10 and 150 N, in particular between 20 and 100 N and, particularly, between 30 and 60 N and disperses in distilled water at 20° C. and/or disintegrates in the mouth in less than 3 minutes, notably in less than 2 minutes, in particular in less than 1 minute and still better in less than 20 sec.

Lastly, the present invention includes a process to prepare an orodispersible and/or dispersible tablet including the steps consisting in:

1) mixing to homogeneity at least one active substance with particle size not exceeding 50 μm, at least one diluent, without known effect et non water-soluble, at least one disintegrating agent, at least one sweetening agent with particle size not exceeding 50 μm such as previously defined and in the proportions previously defined and, possibly, one (or several) other excipient(s),

2) possibly sieving the mixture and/or while preparing said mixture and this, to remove the undesired agglomerates,

3) possibly lubricating said mixture by adding at least one lubrication agent such as previously defined and in the proportions previously defined,

4) subjecting to a direct compression said mixture possibly having been sieved and/or lubricated in a tablet machine provided with adequate punches to obtain an orodispersible and/or dispersible tablet delivering the desired quantity of active substance.

By “mixing to homogeneity”, one understands, according to the European and American Pharmacopeia, mixing in order to obtain a distribution of the active substance within the mixture allowing to guarantee a coefficient of variation of the dosage of the aforementioned substance, as a measurement of uniformity of content, not exceeding 6%.

The possible other excipients added to the mixture of the process of the invention comprise notably the excipients previously described and, in particular, the flowing agents, flavouring agents, colouring agents and humectants such as previously defined and in the proportions previously defined.

The mixture of the ingredients (active agent and excipients) can be carried out by adding these ingredients either simultaneously, or sequentially. The addition in a sequential way of the ingredients makes it possible to introduce one or more steps of sieving and/or lubrication during the step of mixture.

In a first form of implementation of the method of preparation according to the invention, the step (1) of the latter can include the steps consisting in:

a) mixing at least one active substance with particle size not exceeding 50 μm such as previously defined and in the proportions previously defined with at least one flowing agent, without known effect and non water-soluble, such as previously defined and in the proportions previously defined,

b) possibly, sieving the mixture of step (a),

c) adding at least one diluent such as previously defined then mixing,

d) possibly, sieving the mixture of step (c),

e) adding to the possibly sieved mixture of step (d), at least one diluent such as previously defined, at least one disintegrating agent such as previously defined and in the proportions previously defined, at least one sweetening agent with particle size not exceeding 50 μm such as previously defined and in the proportions previously defined and, possibly, at least one colouring agent such as previously defined and in the defined proportions then mixing and at least one flavouring agent such as previously defined and in the proportions previously defined,

the diluents of steps (c) and (e) identical or different being such that the final proportion of the diluent(s) in the final tablet is 40 to 99% by weight compared to the total weight of the tablet.

This first form of implementation is in particular that which is illustrated on FIG. 4.

In a second form of implementation of the method of preparation according to the invention, the step (1) of the latter can include the steps consisting in:

a′) mixing at least one active substance such as previously defined and in the proportions previously defined with at least one flowing agent such as previously defined and in the proportions previously defined,

b′) possibly, sieving the mixture of step (a′),

c) adding at least one diluent such as previously defined then mixing,

d′) possibly, sieving the mixture of step (c),

e′) adding at least one diluent such as previously defined then mixing,

f′) possibly, sieving the mixture of step (e′),

g′) adding to the possibly sieved mixture of step (f′), at least one diluent such as previously defined, at least one disintegrating agent such as previously defined and in the proportions previously defined, at least one sweetening agent with particle size not exceeding 50 μm such as previously defined and in the proportions previously defined and, possibly, at least one colouring agent such as previously defined and in the defined proportions then mixing and at least one flavouring agent such as previously defined and in the proportions previously defined, the diluents of steps (c), (e′) and (g′) identical or different being such that the final proportion of diluent in the final tablet is 40 to 99% by weight compared to the total weight of the tablet.

The mixture obtained at the end of steps (e) or (W) is subjected to steps 2, 3 and 4 of the process such as previously defined. The various ingredients added at the steps (c), (e), (c′), (e′) and (g′) of the processes can be subjected to a sieving before their addition.

The two forms of implementation previously described can present alternatives.

Therefore, a first alternative (i) relates to step (a) and step (a′). Indeed, one can consider that said at least one active substance such as previously defined and in the proportions previously defined is mixed with a given quantity of diluent before its mixture with at least a flowing agent such as previously defined and in the proportions previously defined. The only condition with this alternative is:
within the scope of the first form of implementation previously defined, that the diluent used in this alternative and the diluents of steps (c) and (e), identical or different, are such that the final proportion of diluent (a) in the final tablet is 40 to 99% by weight compared to the total weight of the tablet;
within the scope of the second form of implementation previously defined, that the diluent used in this alternative and the diluents of steps (c′), (e′) and (W), identical or different, are such that the final proportion of diluent(s) in the final tablet is 40 to 99% by weight compared to the total weight of the tablet.

A second alternative relates to step (e) and step (g′). Indeed, one can consider that the added ingredients, at the time of these steps, respectively to the mixtures of steps (d) and (f′) are added

(ii) together after being mixed all together with a diluent such as previously defined then possibly sieved,

(iii) one after the other, each one having been mixed beforehand with a diluent such as previously defined then possibly sieved,

(iv) together after being mixed all together with a diluent such as previously defined then possibly sieved, certain ingredients having been mixed beforehand with a diluent such as previously defined then possibly sieved.

FIG. 1 illustrates a method of preparation presenting the alternative (iii) for step (g′) and FIG. 3 illustrates the alternative (ii) for step (g′) and FIG. 4 illustrates the alternative (iv) for step (e) FIG. 6 illustrates a method of preparation with, at the same time, the alternative (i) for step (a) and the alternative (iii) for step (e).

The choice of the punches of the machine for compression, of their shape and their size, the operating conditions during possible sievings or compression is routine work for one skilled in the art, just as the choice of the size of the sieve to be implemented according to the nature of the ingredient or the mixture of ingredients to be sieved.

Surprisingly, it was highlighted that the qualitative and quantitative composition and its manufacturing process, according to the invention, made it possible to obtain an in vitro dissolution profile faster than the marketed forms.

Therefore, thanks to the solid pharmaceutical composition and its manufacturing process, according to the invention, one can prepare orodispersible and/or dispersible tablets which have an in vitro dissolution profile in a buffer solution at pH 1.2 with more than 75% of active substance released in less than 5 min and in particular more than 90% of active substance released in less than 5 min.

More surprisingly, it was highlighted that the qualitative and quantitative composition and its manufacturing process, according to the invention, made it possible to obtain in vivo pharmacokinetic profiles comparable with a marketed orodispersible composition such as for example oral lyophilizates.

Other characteristics and advantages of this invention will still appear on reading of the examples given hereafter on a purely illustrative and non-restrictive basis and referring to the figures appended.

### BRIEF DESCRIPTION OF THE FIGURES

FIG. 1 shows the diagram of manufacture implemented for formulations containing risperidone (D2 dopamine antagonist with a preferential action at the level of the limbic system) as an active ingredient and in conformity with a first alternative of the method of preparation such as previously defined.

FIG. 2 shows the in vitro dissolution profiles obtained for formulations according to the invention and marketed oral lyophilizates containing as active ingredient either 0.5 mg, or 2 mg of risperidone.

FIG. 3 shows the diagram of manufacture implemented for formulations containing donepezil (inhibitor of cholinesterase which, by increasing at the central level the acetylcholine concentrations in the synaptic cleft, would improve the cognitive function during Alzheimer's disease) as an active ingredient and in conformity with a second alternative of the method of preparation such as previously defined.

FIG. 4 shows the diagram of manufacture implemented for formulations containing donepezil as an active ingredient and in conformity with a third alternative of the method of preparation such as previously defined.

FIG. 5 shows the in vitro dissolution profiles obtained for formulations according to the invention containing as active ingredient either 5 mg, or 10 mg of donepezil and for a speciality marketed with 10 mg of donepezil.

FIG. 6 shows the diagram of manufacture implemented for formulations containing nebivolol (beta-blocking cardioselective indicated in the treatment of hypertension) as an active ingredient and in conformity with a fourth alternative of the method of preparation such as previously defined.

### DETAILED DESCRIPTION OF PARTICULAR EMBODIMENTS

### Example 1

### Orodispersible/Dispersible Compositions of Risperidone Obtained by a Mixing Process by Dilution/Direct Compression

A. Choice of Excipients

The selected excipients, within the scope of example 1 and example 2 and, usually, within the scope of this invention, guarantee an industrial feasibility, a stable end product and without danger to the patient (use of excipients without known effect recorded with the Pharmacopoeia).

Microcrystalline cellulose: This excipient is a diluent usually used in the manufacture of tablets. Several types of microcrystalline cellulose are available. They differ by their manufacturing process, the size of the particles, residual moisture, the flow or other physical properties. For the development of an orodispersible/dispersible tablet, the choice was made of Vivapur® 12 marketed by the company JRS. Indeed, it has a residual moisture less than 5%, a good flow, a particle size distribution adapted to the other components (average size of the particles of 180 μm) and with a good aptitude for direct compression. Any supply of equivalent quality can replace it.

Sodium Croscarmellose (or cross-linked carboxymethylcellulose): This excipient is a disintegrating agent generally used in dry mixture to a total value of 2 to % in the formula. The sodium croscarmellose used during the development is AcDiSol° marketed by the company FMC Biopolymer. Any supply of equivalent quality can replace it.

Potassium acesulfame: This intense sweetening substance is frequently employed in pharmaceutical preparations. The potassium acesulfame improves flavours and accentuates the effect of flavouring agent(s) used to mask the bad taste of the active substance. The potassium acesulfame used during the development is provided by ACT. Any supply of equivalent quality can replace it.

Peppermint flavour: A flavouring of mint type was selected from order to mask the unpleasant taste of the active substance. The peppermint flavour used during development is provided by IFF. Any supply of equivalent quality can replace it.

Hydrophobic colloidal silica: This excipient is used in the formula as a flowing agent in order to allow a good distribution of the active substance. Therefore, the use of colloidal silica makes it possible to correctly distribute the active substance in the mixture. Colloidal silica used is provided by the company DEGUSSA under the denomination Aerosil® 200. Any supply of equivalent quality can replace it.

Magnesium stearate: This excipient is usually used in pharmaceutical preparations for its lubricating properties. The magnesium stearate of vegetable origin used during the development is provided by the company PETER GREVEN under the denomination Liga® MF2V. Any supply of equivalent quality can replace it.

Dye E172: This natural pigment of an iron oxide type makes it possible to obtain a composition with a pink colouring. The dye E172 used during the development is provided by the company BASF under the denomination Sicovit® Rouge 30.

B. Compositions and Specifications of the Formulas Carried Out

Table 1 hereafter shows, for each formulation carried out within the scope of this invention, its composition and its specifications.

TABLE 1

| | mg/unit | | | | | |
|---|---|---|---|---|---|---|
| Composition (%) | 1 | 2 | 3 | 4 | 5 | %/unit |
| Risperidone (API) | 0.500 | 1.000 | 2.000 | 3.000 | 4.000 | 1.00 |
| Hydrophilic colloidal silica | 0.005 | 0.010 | 0.020 | 0.030 | 0.040 | 0.01 |
| Microcristalline cellulose | qs | qs | qs | qs | qs | qs |
| Crosslinked carboxymethycellul. | 2.500 | 5.000 | 10.000 | 15.000 | 20.000 | 5.00 |
| Peppermint flavour | 0.625 | 1.250 | 2.500 | 3.750 | 5.000 | 1.25 |
| Potassium acesulfame | 1.000 | 2.000 | 4.000 | 6.000 | 8.000 | 2.00 |
| Dye E172 | 0.010 | 0.020 | 0.040 | 0.060 | 0.080 | 0.02 |
| Magnesium stearate | 0.250 | 0.500 | 1.000 | 1.500 | 2.000 | 0.50 |
| Checking final mixtures | | | | | | |
| Residual moisture (%) | 4.5-5.5 | | | | | |
| Flow (s) | <10 | | | | | |
| Volume mass in bulk | 0.35-0.55 | | | | | |
| Aspect | Pink powder | | | | | |
| Checking finished product | | | | | | |
| Punch Format | Flat | Flat | Flat. Ø | Flat. Ø | Flat. Ø | |
| | bevelled | bevelled | 9R11 mm | 10R12 mm | 11R11 mm | |
| | edge 30°. | edge 30°. | | | | |
| | Ø 5.5 mm | Ø 6.5 mm | | | | |
| Thickness | 1.75-2.15 | 2.50-2.90 | 3.45-3.35 | 4.30-4.70 | 5.25-5.65 | |
| Breaking strength (N) | 30-50 | 45-65 | 45-65 | 55-75 | 55-75 | |
| Disintegration - Water R 37° C. | <10 | <10 | <10 | <10 | <10 | |
| (s) | | | | | | |
| Dispersion - Water R 20° C. (s) | <15 | <15 | <15 | <15 | <15 | |
| Smoothness of dispersion | Compliant | Compliant | Compliant | Compliant | Compliant | |
| Friability (%) | <0.5 | <0.5 | <0.5 | <0.5 | <0.5 | |
| Uniformity of content | Compliant | Compliant | Compliant | Compliant | Compliant | |
| Dissolution in vitro | 75% | 75% | 75% | 75% | 75% | |
| (at 15 min in buffer pH 1.2) | | | | | | |
| Theoretical mass of tablet (mg) | 50.00 | 100.00 | 200.00 | 300.00 | 400.00 | |

C. Manufacturing Process

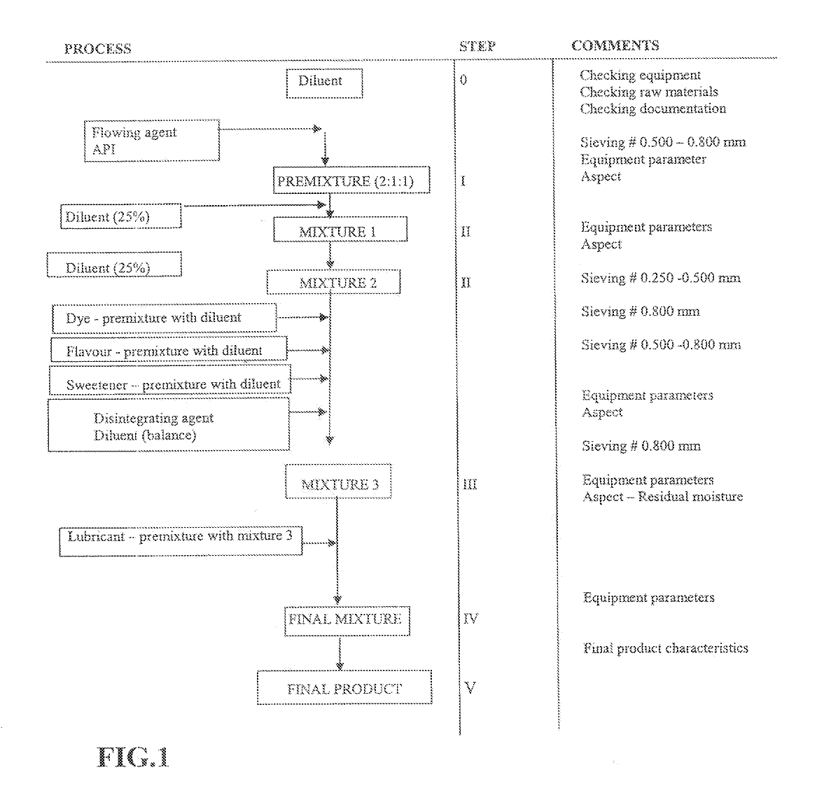

FIG. 1 shows the diagram of manufacture implemented for the formulations containing risperidone.

Step 1: Premixture

Mixing of the whole quantity of flowing agent with the totality of the risperidone (API) and 25% of the quantity of diluent in polyethylene bags, turned upside-down 10 times.

Step 2: Mixture 1

Introducing into a Roue Rhöen mixer, 25% of the quantity of diluent then the premixture beforehand sieved on a grid with a mesh-size of 800 μm.

Mixing the unit over 5 min at approximately 10 rpm.

Step 3: Mixture 2

Adding to mixture 1 the balance of the diluent.

Mixing the unit over 5 min at approximately 10 rpm.

Step 4: Mixture 3

Adding to mixture 2, the premixed dye with diluent and sieved on a grid with a mesh-size ranging between 0.250 and 0,500 mm, the premixed flavour with diluent and sieved on a grid with a mesh-size of 0.800 mm, the sweetening agent premixed with diluent and sieved on a grid with a mesh-size ranging between 0.500 and 0.800 mm, the disintegrating agent premixed with diluent then the balance of diluent.

Mixing the unit over 15 min at approximately 10 rpm.

Step 5: Final Mixture

Adding to mixture 3 the lubricant sieved beforehand on a grid with a mesh-size of 0.800 mm.

Mixing 5 min at approximately 10 rpm.

Step 6: Compression

Equipping the machine to compress with suitable punches and regulating it in order to obtain the characteristics of the tablets described according to the invention.

Step 7: Packaging

Packaging the tablets in thermoformed blisterpack PVC-PVDC/Aluminium.

D. Study of Compared In Vitro Dissolution

The operating conditions implemented for this study are as follows:

Equipment Dissolutest: Sotax AT7 spectrophotometer Lambda 20

Method: HPLC

Dosed compound: Risperidone

Chamber volume: 1000 ml

Medium Buffer: HCl 0.1N

Rotation of the axis: 50 rpm

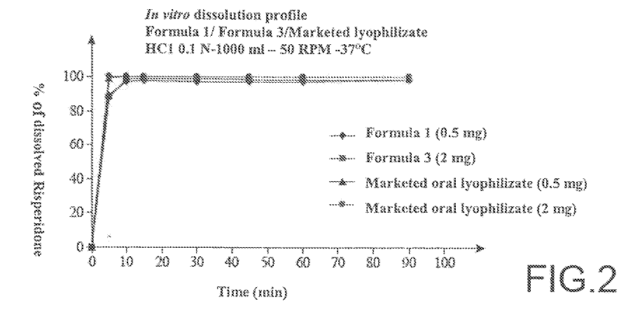

The in vitro dissolution profiles obtained for two formulations according to the invention and two marketed oral lyophilizates containing risperidone (Risperdal® Oro 0.5 mg and 2 mg) are shown on FIG. 2.

### Example 2

### Orodispersible/Dispersible Compositions of Donepezil Obtained by a Mixing Process by Dilution/Direct Compression

A. Choice of Excipients

Microcrystalline cellulose: This excipient is a diluent usually used in the manufacture of tablets. Several grades are available, the choice was led to Vivapur® 14 marketed by the company JRS. Vivapur® 14 is equivalent to grade 12 but with very low moisture content, less than 1.5%. This excipient is particularly adapted for active ingredients or compositions sensitive to moisture. Any supply of equivalent quality can replace it.

Sodium Croscarmellose: cf Example 1.

Pregelatinized corn starch: This excipient is a disintegrating agent used for the preparation of tablets. For the development of an orodispersible/dispersible tablet, the choice was made of Starch 1500 marketed by the company COLORCON. Thanks to its rheological properties (flow, particle size . . . ), this excipient is particularly adapted for direct compression. Any supply of equivalent quality can replace it.

Sodium starch glycolate: This effective and inexpensive disintegrating agent is particularly adapted for direct compression. The sodium starch glycolate used during the development of the composition is provided by JRS Pharma under the trade description Explotab®. Any supply of equivalent quality can replace it.

Potassium Acesulfame: cf Example 1.

Sodium saccharin: Sodium saccharin as an intense sweetener used in drinks, foodstuffs, table sweetening substances and pharmaceutical formulations. Sodium saccharin is considerably more water soluble than saccharin and also more frequently employed in pharmaceutical formulations. Its sweetening capacity is roughly 300 times more powerful than sugar. Sodium saccharin improves flavours and can be employed to mask certain unpleasant characteristics of taste. All these characteristics are in conformity with the objective to develop an orodispersible and/or dispersible tablet. The Laboratory COOPER provides the sodium saccharin used during the development. Any supply of equivalent quality can replace it.

Peppermint Flavour: cf Example 1.

Hydrophobic anhydrous colloidal silica: This excipient is used in the formula as flowing agent in order to allow a good distribution of the active substance. Therefore, the use of colloidal silica makes it possible to correctly distribute the active substance in the mixture Colloidal silica used is provided by the company DEGUSSA under the denomination Aerosil® R972. This quality has hydrophobic properties and makes it possible for the orodispersible and/or dispersible tablet to be less sensitive to the phenomenon of resumption of moisture. Any supply of equivalent quality can replace it.

Magnesium Stearate: cf Example 1,

Dye E172: cf Example 1.

B. Compositions and Characteristics of the Formulas Carried Out

Table 2 hereafter shows, for each formulation carried out within the scope of this invention, its composition and its characteristics.

TABLE 2

| | Composition (%) | | | |
|---|---|---|---|---|
| | Formula n° | Formula n° | Formula n° | Formula n° |
| Components | 6 | 7 | 8 | 9 |
| Donepezil | 3.57 | 3.57 | 2.04 | 3.57 |
| Cellulose | 88.78 | 88.78 | Qs | Qs |
| microcrystalline with low | | | | |
| moisture content | | | | |
| Pregelatinized corn | — | 5.00 | — | — |
| starch | | | | |
| Potassium acesulfame | — | 1.00 | 1.00 | 1.00 |
| AcDiSol | 5.00 | — | 2.50 | 2.50 |
| Explotab | — | — | 2.50 | 2.50 |
| Sodium saccharine | 1.00 | — | — | — |
| Dye E172 | 0.05 | 0.05 | — | 0.50 |
| Peppermint aroma | 1.00 | 1.00 | 0.75 | 0.75 |
| Hydrophobic colloidal | 0.10 | 0.10 | 0.10 | 0.10 |
| silica | | | | |
| Magnesium stearate | 0.50 | 0.50 | 0.50 | 0.50 |
| Checking final mixture | | | | |
| Flow (s) | <10 | <10 | <10 | <10 |
| Checking final product | | | | |
| Format for marking | Flat. Ø | Flat. Ø | Flat. Ø | Flat. Ø |
| instrument | 9.5 mm | 9.5 mm | 9 mm | 9 mm |
| Breaking strength (N) | 48 | 61.20 | 71.80 | 44.50 |
| Disintegration - | 15 | 13 | 4 | 7 |
| Water R 37° C. - (s) | | | | |
| Dispersion - | <10 | <10 | <10 | <10 |
| Water R 20° C. - (s) | | | | |
| Smoothness of dispersion | compliant | compliant | compliant | compliant |
| Friability (%) | 0.31 | 0.05 | <0.01 | <0.01 |
| Theoretical mass of | 280 | 280 | 245 | 280 |
| tablet (mg) | | | | |

C. Manufacturing Process

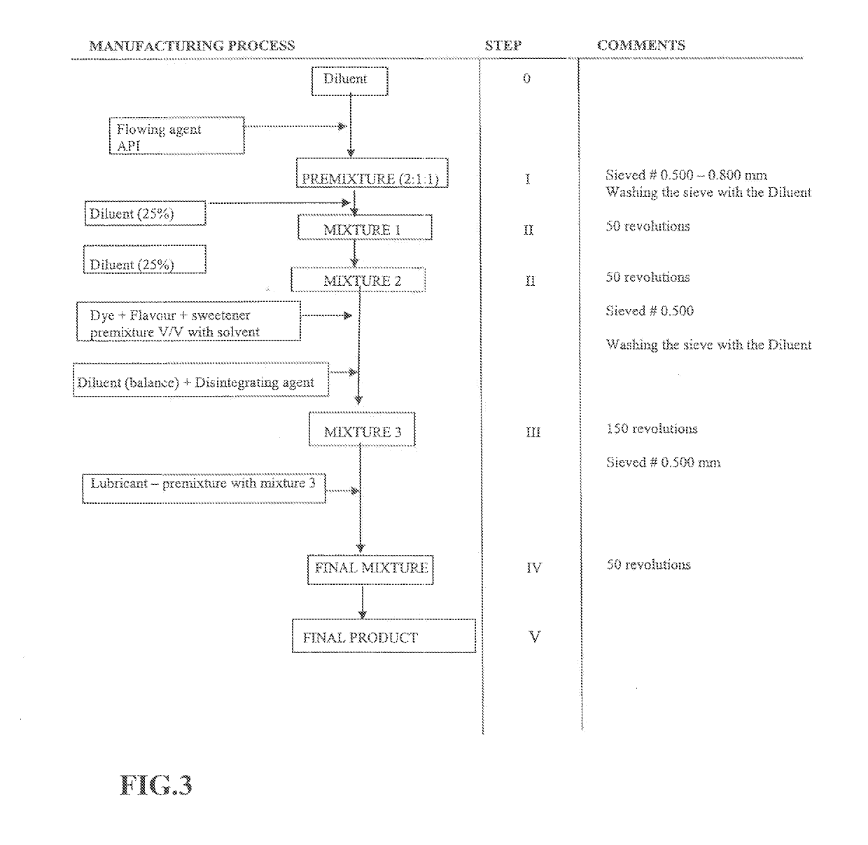

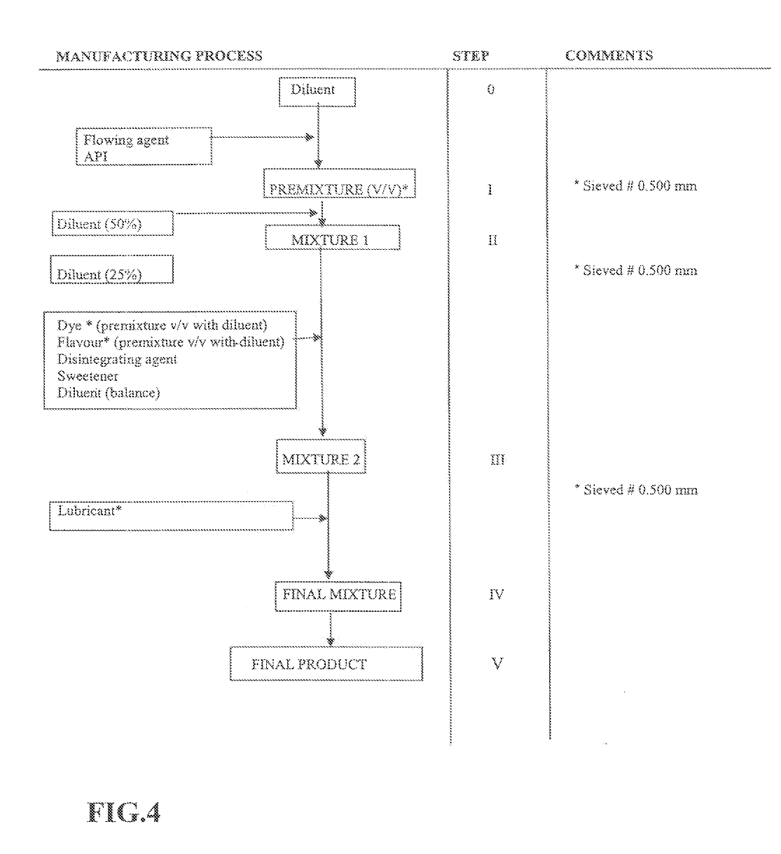

FIGS. 3 and 4 show two diagrams of manufacture implemented for formulations containing donepezil.

The preparation method object of FIG. 3 is described more in detail hereafter.

Step 1: Premixture

Mixing of the whole quantity of flowing agent with the totality of donepezil (API) and 25% of the quantity of diluent in polyethylene bags, turned upside-down 10 times.

Step 2: Mixture 1

Introducing into a Roue Rhöen mixer, 25% of the quantity of the diluent then the premix sieved beforehand grid with a mesh-size of 0.500 mm

Mixing the unit during 5 min at approximately 10 rpm.

Step 3 Mixture 2

Adding to mixture 1 the balance of the diluent.

Mixing the unit during 5 min at approximately 10 rpm.

Step 4 Mixture 3

Adding to mixture 2, the dye, the flavour, the sweetening agent premixed with diluent and sieved on a grid with a mesh-size of 0500 mm then the balance of diluent and the disintegrating agent.

Mixing the unit over 15 min at approximately 10 rpm.

Step 5: Final Mixture

Adding to mixture 3 the lubricant beforehand sieved on a grid with a mesh-size of 0.500 mm.

Mixing 5 min at approximately 10 rpm.

Step 6: Compression

Equipping the machine to compress with suitable punches and to regulate it in order to obtain the characteristics of the tablets described according to the invention.

Step 7: Packaging

Packaging the tablets in thermoformed blisterpack PVC-PVDC/Aluminium.

The preparation method object of FIG. 4 differs from the process above from the fact that steps 1 and 2 are carried out simultaneously by mixing the flowing agent and the totality of donepezil (Aricept®) with 50% of diluent.

D. Study of Compared In Vitro Dissolution

The operating conditions implemented for this study are as follows:

Equipment: Dissolutest Sotax AT7 spectrophotometer Lambda 20

Method: HPLC

Dosed compound: Donepezil

Chamber volume: 1000 ml

Medium: Buffer pH 1.2

Rotation of the axis: 75 rpm

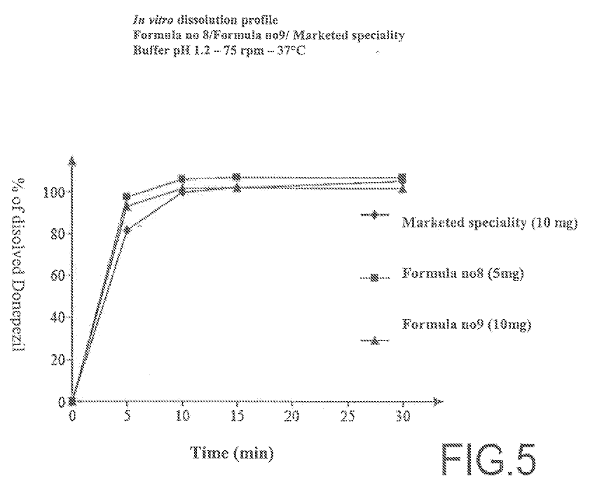

The in vitro dissolution profiles obtained for two formulations according to the invention containing as active ingredient either 5 mg, or 10 mg of donepezil and for a marketed speciality with 10 mg of donepezil are shown on FIG. 5.

### Example 3

### Orodispersible/Dispersible Compositions of Nebivolol Obtained by a Mixing Process by Dilution/Direct Compression

A. Choice of Excipients

The excipients shown in the formulation containing Nebivolol are described in example 2.

B. Compositions and Characteristic of the Formulas Carried Out

Table 3 hereafter shows, for the formulation carried out within the scope of this invention, its composition and its specifications. This composition is applicable whatever the proportioning desired (1.25; 2.5; 5; 10 mg . . . ).

TABLE 3

| Components | Composition (%) |
|---|---|
| Nebivolol | 2.50 |
| Cellulose microcrystalline with low | Qs |
| moisture content | |
| Potassium acesulfame | 1.25 |
| AcDiSol | 5.00 |
| Peppermint aroma | 2.00 |
| Hydrophobe colloidal silica | 0.10 |
| Magnesium stearate | 0.50 |
| Checking final mixture | |
| Flow (s) | <10 |

TABLE 4

| | Formula n° 10 | Formula n° 11 |
|---|---|---|
| Checks | (1.25 mg) | (10 mg) |
| Average mass (mg) | 51.96 | 404.50 |
| Content uniformity | Compliant | Compliant |
| Breaking strength (N) | 35 | 75 |
| Disintegration - Water | 7 s | 10 s |
| R 37° C.(s)) | | |
| Dimensions | | |
| Thickness (mm) | 1.78 | 3.48 |
| Diameter (mm) | 5.56 | 11.44 |

C. Manufacturing Process

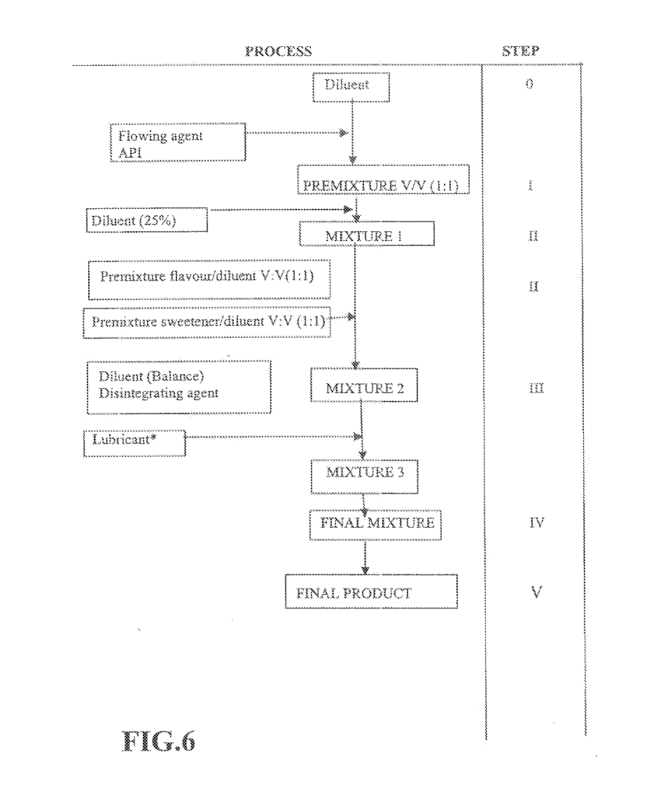

FIG. 6 presents a diagram of manufacture implemented for formulations containing nebivolol.

The method of object preparation of FIG. 6 is described more in detail hereafter.

Step 1: Premixture

Mixing the totality of Nebivolol (API) with an equivalent volume of diluent in a standard Roue Rhöen mixer.

Step 2 Mixture 1

Adding the flowing agent (glidant) on the premixture in a Roue Rhöen mixer.

Mixing the unit over 5 min at approximately 10 rpm.

Step 3: Mixture 2

Adding to the mixture 1, 25% of diluent,

Mixing the unit over 5 min at approximately 10 rpm.

Step 4 Mixture 3

Adding to mixture 2, the premixed flavour with an equivalent volume of diluent and sieved on a grid with a mesh-size of 0.500 mm, the sweetening agent also premixed and sieved on a grid with a mesh-size of 0.500 mm with an equivalent volume of diluent then the balance of diluent and the disintegrating agent.

Mixing the unit over 15 min at approximately 10 rpm.

Step 5: Final Mixture

Adding to mixture 3 the lubricant agent previously sieved on a grid with a mesh-size of 0.500 mm

Mixing 5 min at approximately 10 rpm.

Step 6 Compression

Equipping the machine to compress with suitable punches and to adjust it in order to obtain the characteristics of the tablets described according to the invention.

Step 7: Packaging

Packaging the tablets in thermoformed blisterpack PVC-PVDC/Aluminium.

1. A solid, orodispersible and/or dispersible composition, without an excipient of known effect, comprising
a) from 0.1 to 59 a by weight of at least one active substance with particle size not exceeding 50 μm,
b) from 40 to 99% by weight of at least one diluent without known effect, non water-soluble;
c) from 0.1 to 15% by weight of at least one disintegrating agent; and
d) from 0.05 to 10% by weight of at least one sweetening agent with particle size not exceeding 50 μm,
the percentages by weight being expressed compared to the total weight of the aforementioned composition.

2. The composition according to claim 1, wherein, the aforementioned active substance is a pharmaceutical active ingredient, a parapharmaceutical active ingredient, a cosmetic active ingredient, a neutraceutical active ingredient, a food ingredient or an agroalimentary supplement.

3. The composition according to claim 1, wherein the aforementioned active substance belongs to one of the following pharmacotherapeutic families: allergology, anaesthesia/resuscitation, cancerology and haematology, cardiology and angiology, contraception and termination of pregnancy, dermatology, endocrinology, gastro-entero-hepathology, gynaecology and obstetrics, immunology and transplantation drugs, study of infections and parasitology, metabolism, diabetes and nutrition, neurology/Psychiatry, ophthalmology, otorhinolaryngology, pneumology, rheumatology, stomatology, toxicology, urology nephrology, as well as among analgesic/anti-pyretic and antispasmodic drugs, anti-inflammatory drugs, the products of diagnosis, haemostatics and blood treatment products and derivatives.

4. The composition according to claim wherein the aforementioned active substance is selected from the group consisting of an emollient, a wetting agent, a pigment, a dye, an anti-wrinkle agent, an anti-fungal agent, an anti-acne agent, a softening agent, a perfume, a vitamin and mixtures thereof.

5. The composition according to claim 1, wherein the aforementioned active substance is selected from the group consisting of a vitamin, a mineral, a carotenoid, a phyto-oestrogen, a vegetable extract, a clay, a ferment, a yeast and their mixtures.

6. The composition according to claim 1, wherein the aforementioned diluent is selected from the group consisting of a cellulose powder, a non-silicified microcrystalline cellulose, a microcrystalline cellulose with low water content, a cellulose acetate, a di-basic calcium phosphate, a tri-basic calcium phosphate, a calcium sulphate, a dextrate, a dextrin, a hydrogenated vegetable oil, a glyceryl palmitostearate, and a polymethacrylate.

7. The composition according to claim 1, wherein the aforementioned disintegrating agent is selected from the group consisting of crospovidone, croscarmellose, sodium starch glycolate, sodium alginate, hydroxypropyl cellulose, starch, pregelatinized starch, derivatives and mixtures thereof.

8. The composition according to claim 1, wherein the aforementioned sweetening agent is selected from among the sugar substitutes and, more particularly among gluconate, potassium acesulfame, sodium saccharinate, lithium saccharinate, cyclamate, or mixtures thereof.

9. The composition according to claim 1, wherein the aforementioned composition comprises from 0.005 to 10% by weight of a flavouring agent or a mixture of flavouring agents, compared to the total weight of the composition.

10. The composition according to claim 1, wherein the aforementioned composition comprises from 0.001 to 1% by weight of one or several flowing agents, compared to the total weight of the composition.

11. The composition according to claim 1, wherein the aforementioned composition comprises from 0.01 to 2% by weight of a lubricant agent or a mixture of lubricant agents, compared to the total weight of the composition.

12. The composition according to claim 1, wherein the aforementioned composition comprises from 0.01 to 5% by weight of a humectant or a mixture of humectants, compared to the total weight of the composition.

13. The composition according to claim 1, for use as a drug, a food supplement or in cosmetics.

14. A method of preparing an orodispersible and/or dispersible tablet comprising:
(1) mixing to homogeneity at least one active substance with particle size not exceeding 50 μm, at least one diluent without known effect, non water-soluble, at least one disintegrating agent, at least one sweetening agent with particle size not exceeding 50 μm such as defined in claim 1 and, optionally, one or more other excipients,
(2) optionally sieving the mixture and/or while preparing said mixture,
(3) optionally lubricating said mixture adding at least one lubricant agent as defined in claim 11,
(4) subjecting to a direct compression said mixture possibly having been sieved and/or lubricated in a tablet machine equipped with adequate punches to obtain an orodispersible and/or dispersible tablet delivering the desired quantity of active substance.

15. The process according to claim 14, comprising
(a) mixing at least one active substance with at least one flowing agent,
(b) optionally sieving the mixture of step (a),
(c) adding at least one diluent such as defined in claim 1 then mixing,
(d) optionally sieving the mixture of step (c),
(e) adding to the optionally sieved mixture of step (d), at least one diluent such as defined in claim 1, at least one disintegrating agent such as defined in claim 1, at least one sweetening agent with particle size not exceeding 50 μm such as defined in claim 1,
the diluents of steps and (e), identical or different, being such that the final proportion of diluent in the final tablet is 40 to 99% by weight compared to the total weight of the tablet.

16. The process according to claim 14, comprising:
(a′) mixing at least one active substance such as defined in claim 1, with at least one flowing agent,
(b′) optionally sieving the mixture of step (a′),
(c′) adding at least one diluent such as defined in claim 1 then mixing,
(d′) optionally sieving the mixture of step (c),
(e′) adding at least one diluent such as defined in claim 1 then mixing,
(f′) optionally sieving the mixture of step e′),
(g′) adding to the optionally sieved mixture of step (f′), at least one diluent such as defined in claim 1, a least one disintegrating agent such as defined in claim 1, at least one sweetening agent with particle size not exceeding 50 μm such as defined in claim 1,
the diluents of steps (c′), (e′) and (g′), identical or different, being such that the final proportion of diluent in the final tablet is 40 to 99% by weight compared to the total weight of the tablet.

17. The process according to claim 15, wherein the active substance is a pharmaceutical active ingredient, a parapharmaceutical active ingredient, a cosmetic active ingredient, a neutraceutical active ingredient, a food ingredient or an agroalimentary supplement.
